(19) United States
(12) Patent Application Publication
(10) Pub. No.: US 2010/0268037 A1
(43) Pub. Date: Oct. 21, 2010

(54) EVENT-DRIVEN, DYNAMIC PATIENT SCORECARD

(75) Inventor: Jakka SAIRAMESH, Menlo Park, CA (US)

Correspondence Address: PILLSBURY WINTHROP SHAW PITTMAN LLP, P.O. BOX 10500, MCLEAN, VA, 22102, US

(73) Assignee: 360Fresh, Inc.

(21) Appl. No.: 12/688,669

(22) Filed: Jan. 15, 2010

Related U.S. Application Data
(60) Provisional application No. 61/144,954, filed on Jan. 15, 2009.
(60) Provisional application No. 61/144,962, filed on Jan. 15, 2009.

(51) Int. Cl. A61B 5/00; G06F 17/30

(52) U.S. Cl. 600/300; 707/797; 707/E17.012

(57) ABSTRACT

In general, the invention provides a design and apparatus (patient scorecard system-PSS) for dynamic scoring of Patient clinical condition based on patient-attributes, medical rules, patient-information events and medical criteria. One aspect of the invention is to offer better tracking of Patient-care and improving the quality of care to patients. The scorecard model is based on a multi-attribute data structure that captures Patient risk based on attributes such as symptoms, causes, past history, errors in medication, bad quality of care, medication history, current costs, MRI, current state of the disease and family history. The scorecard keeps a time-stamped record of the attributes of the patients, and updates them dynamically as new information is available from multiple sources such as medical records, MRIs, Lab-tests, personal patient monitors and others.

### CROSS-REFERENCE TO RELATED APPLICATIONS

The present application claims priority to U.S. Provisional Application No. 61/144,954, filed Jan. 15, 2009 and U.S. Provisional Application No. 61/144,962, filed Jan. 15, 2009, the contents of which are incorporated herein by reference in their entirety.

### FIELD OF THE INVENTION

The present invention relates to providing patient health care, and more particularly to a design and apparatus (patient scorecard system-PSS) for dynamic scoring of Patient clinical condition based on patient-attributes, medical rules, patient-information events and medical criteria.

### BACKGROUND OF THE INVENTION

Computerized systems for keeping medical records and monitoring patient care are known. However, many opportunities for improvement remain.

### SUMMARY OF THE INVENTION

In general, the invention provides a design and apparatus (patient scorecard system-PSS) for dynamic scoring of Patient clinical condition based on patient-attributes, medical rules, patient-information events and medical criteria. One aspect of the invention is to offer better tracking of Patient-care and improving the quality of care to patients. The scorecard model is based on a multi-attribute data structure that captures Patient risk based on attributes such as symptoms, causes, past history, errors in medication, bad quality of care, medication history, current costs, MR1, current state of the disease and family history. The scorecard keeps a time-stamped record of the attributes of the patients, and updates them dynamically as new information is available from multiple sources such as medical records, MRIs, Lab-tests, personal patient monitors and others.

In a typical clinical scenario, a Patient makes multiple visits to a Hospital or clinic, and after each visit, new information about the Patient is collected and analyzed for better diagnosis and personalized treatment. In addition any information from the Patient (out-patient) at home or outside the clinic can also be used. The scoring model and mechanism will update the scores of the Patient conditions based on information provided.

The scorecard is updated automatically and dynamically by the scoring-system to ensure that the results are seen in near real-time by anyone authorized to see the Patient treatment, Care and recovery performance. The patient scoring system uses pre-defined rules on patient attributes, criteria and factors to assign weights issued against the attributes in computing a score. The Scoring system checks periodically for information events (e.g. changes in Patient data) from multiple sources. Based on any updates from the Patient or from the medical tests, the scoring system processes the events and generates an updated score and scorecard for the Patient.

### BRIEF DESCRIPTION OF THE DRAWINGS

These and other aspects and features of the present invention will become apparent to those ordinarily skilled in the art upon review of the following description of specific embodiments of the invention in conjunction with the accompanying figures, wherein:

FIG. 1 illustrates a generic patient scorecard system according to example embodiments of the invention.

FIG. 2 illustrates a detailed system design for scoring and learning based on information events occurring during and after Patient-diagnosis or treatment plan according to some aspects of the invention.

FIG. 3 illustrates an example methodology and scoring algorithm for generating the Patient scores according to embodiments of the invention.

FIG. 4 shows an example attribute structure with primary attribute occupying the root of the tree according to aspects of the invention.

FIGS. 5 and 6 are screenshots illustrating example implementations of a tool using aspects of the invention.

### DETAILED DESCRIPTION OF THE PREFERRED EMBODIMENTS

The present invention will now be described in detail with reference to the drawings, which are provided as illustrative examples of the invention so as to enable those skilled in the art to practice the invention. Notably, the figures and examples below are not meant to limit the scope of the present invention to a single embodiment, but other embodiments are possible by way of interchange of some or all of the described or illustrated elements. Moreover, where certain elements of the present invention can be partially or fully implemented using known components, only those portions of such known components that are necessary for an understanding of the present invention will be described, and detailed descriptions of other portions of such known components will be omitted so as not to obscure the invention. Embodiments described as being implemented in software should not be limited thereto, but can include embodiments implemented in hardware, or combinations of software and hardware, and vice-versa, as will be apparent to those skilled in the art, unless otherwise specified herein. In the present specification, an embodiment showing a singular component should not be considered limiting; rather, the invention is intended to encompass other embodiments including a plurality of the same component, and vice-versa, unless explicitly stated otherwise herein. Moreover, applicants do not intend for any term in the specification or claims to be ascribed an uncommon or special meaning unless explicitly set forth as such. Further, the present invention encompasses present and future known equivalents to the known components referred to herein by way of illustration.

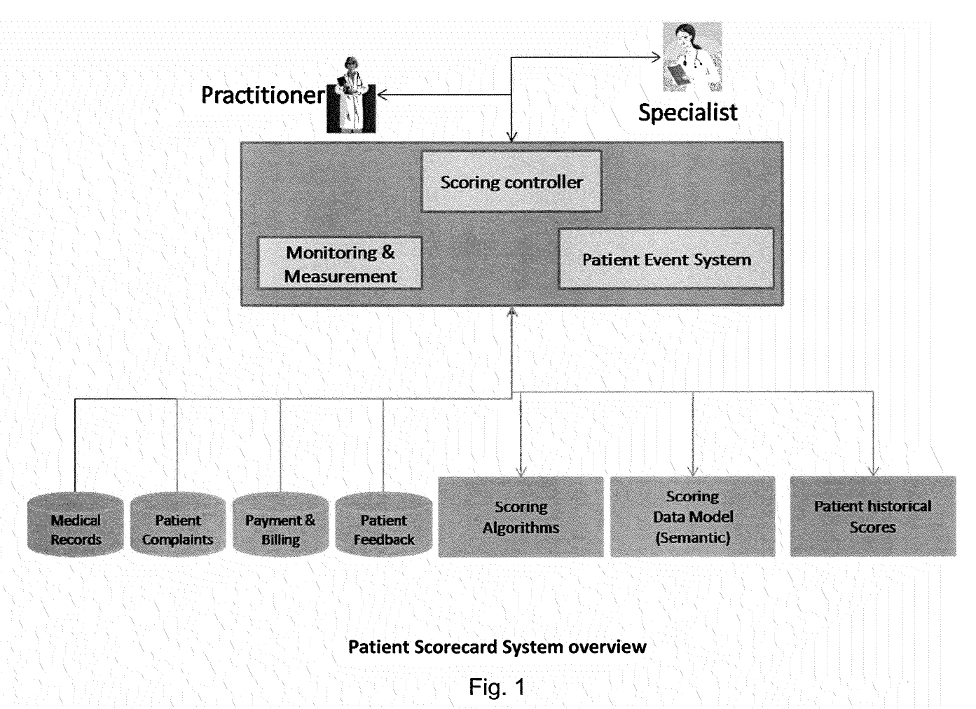

In FIG. 1, we illustrate a generic patient scorecard system according to example embodiments of the invention. As shown, it includes interconnections to patient history, patient record data, and other criteria, and to the scoring components such as an event system, monitoring and scoring controller.

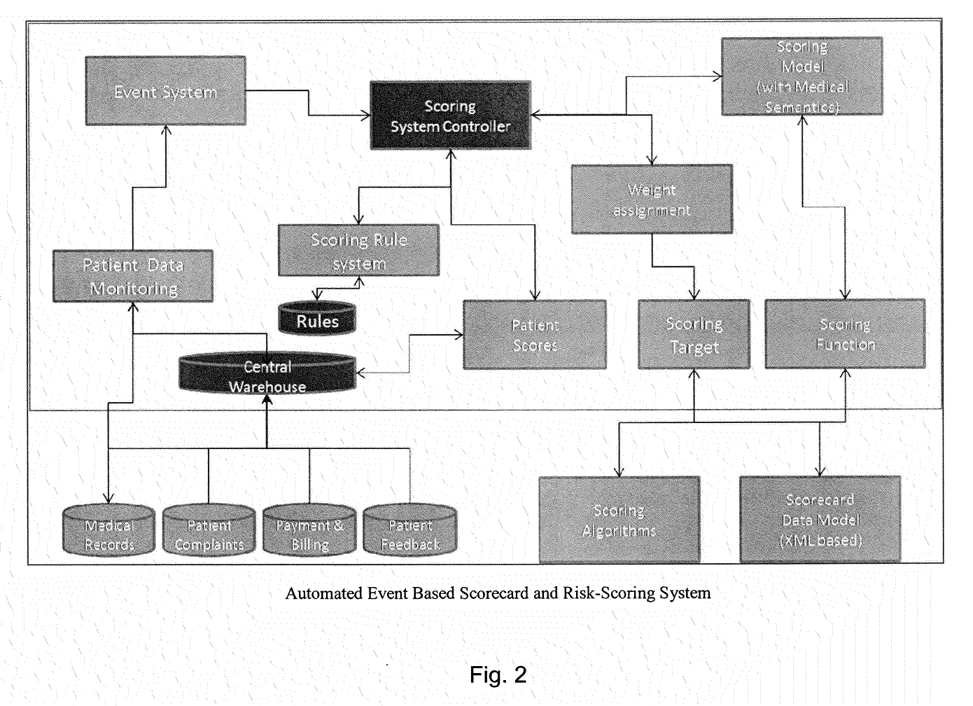

In FIG. 2, we illustrate a detailed system design for scoring and learning based on information events occurring during and after Patient-diagnosis or treatment plan according to some aspects of the invention. The scoring system ensures that events based on new information about a patient are processed, and a score is generated based on the patient attribute (s) that get updated. The scoring-system automatically assigns weights to the attribute based on pre-existing piles.

Scoring Methodology

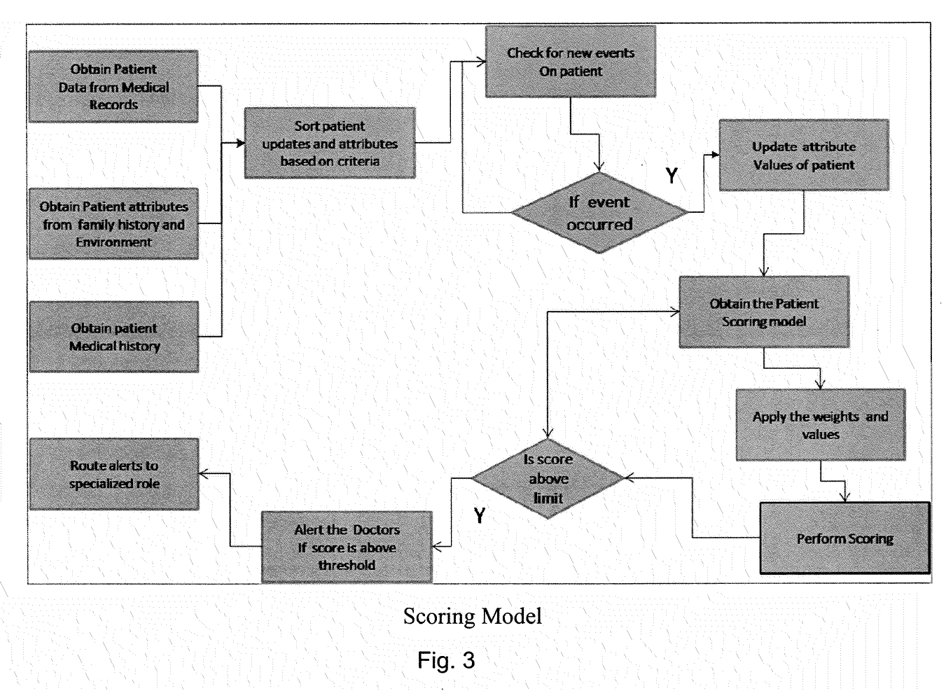

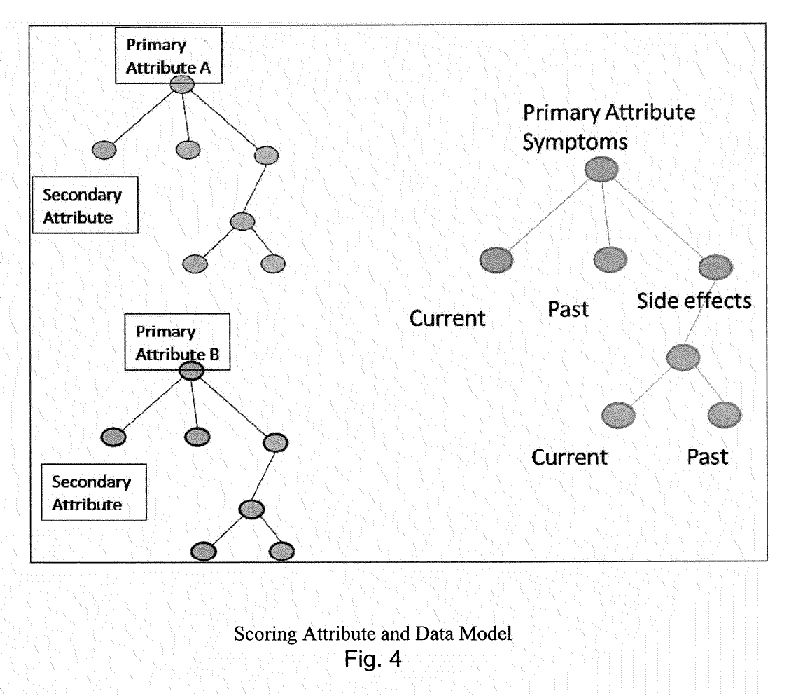

In FIG. 3, we illustrate an example methodology and scoring algorithm for generating the Patient scores according to embodiments of the invention. Events based on the patient data are processed by the Scoring controller (as shown in FIG. 2). The scoring controller follows the algorithm shown in FIG. 3 and uses the data structure shown in FIG. 4, for example. The scoring algorithm checks for new events for every patient in the data warehouse system (FIG. 2) and processes the event as soon as it arrives. The events could also come from changes made to the data in the source systems (e.g. Patient medical records).

Comprehensive Data Model

A comprehensive model for Patient medical scoring based on Patient attributes is considered in this disclosure. The scoring system (as described in FIG. 2) uses an attribute structure that is hierarchical with weights attached to the attributes. FIG. 4 shows the attribute structure with primary attribute occupying the root of the tree.

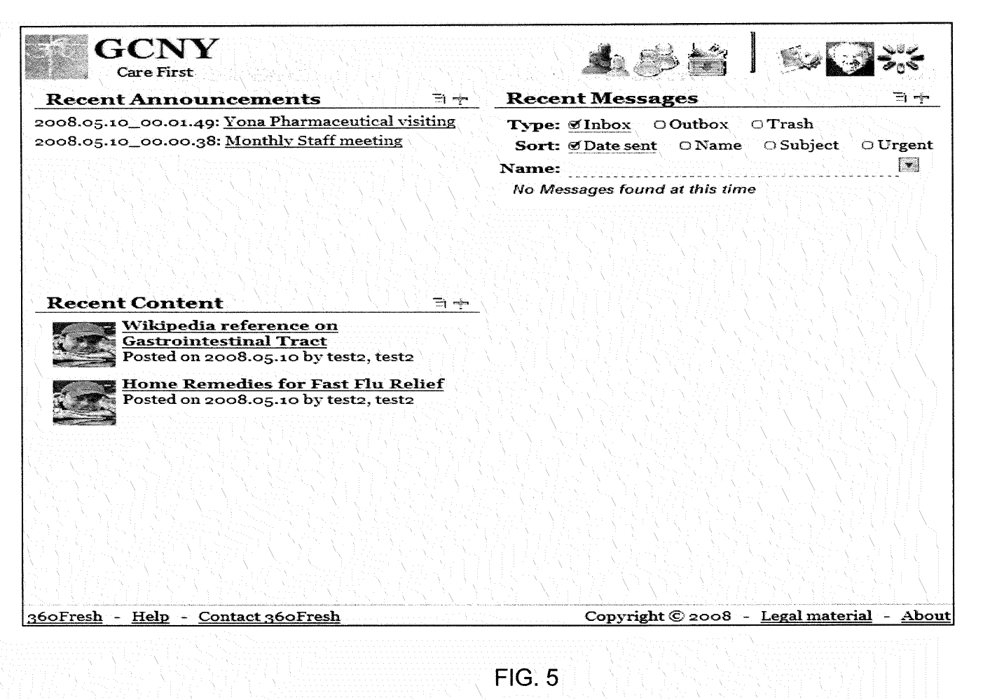

According to certain aspects, the invention provides a Patient Scorecard system to enable dynamic tracking and scoring of Patient treatment and recovery performance based on patient attributes, weights and assessment rules. In FIG. 5 we illustrate an example of the tool workspace for monitoring and scoring patients on side effects, medical issues, quality of care and other criteria.

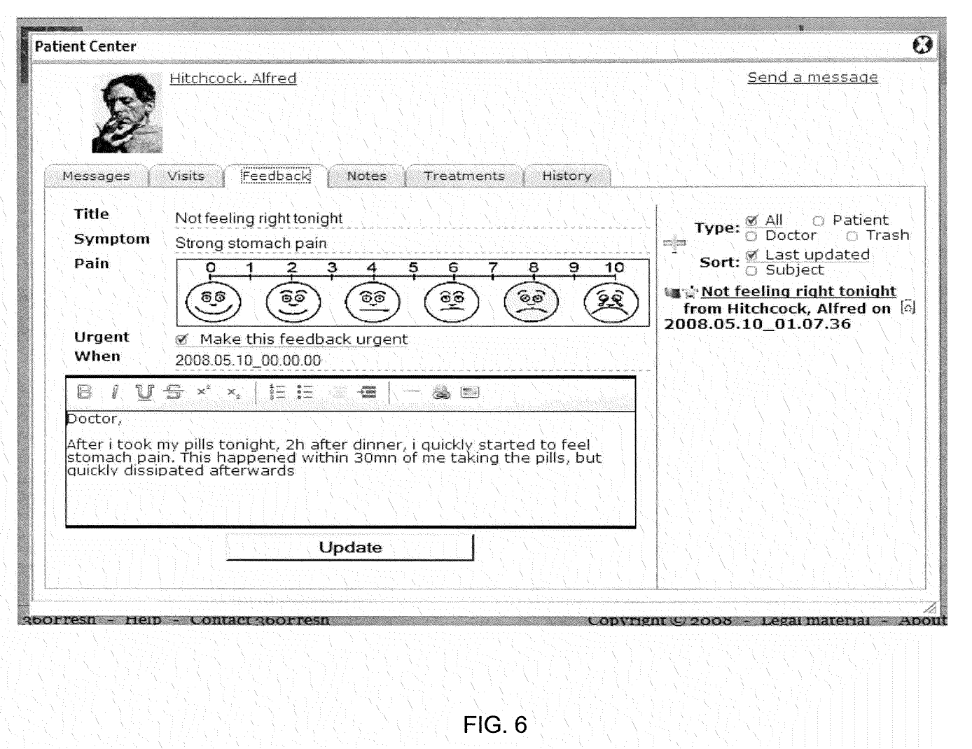

The Scoring system is based on components such as the Pain-Chart shown in FIG. 6, which provides feedback to Doctors on Patient performance, compliance to medication and current symptoms.

Although the present invention has been particularly described with reference to the preferred embodiments thereof, it should be readily apparent to those of ordinary skill in the art that changes and modifications in the form and details may be made without departing from the spirit and scope of the invention. It is intended that the appended claims encompass such changes and modifications.

What is claimed is:

1. An automatic mechanism and generic system (PSS) for dynamic scoring of patient attributes based on information events generated by changes in Patient records, tests, images, medication history, family history, genetic history and daily'symptoms.

2. A hierarchical (tree) scorecard model for patient data based on patient attributes.

3. A model according to claim 2, further including the dynamic assignment of weights to the tree based scorecard model.

4. An automated learning and monitoring mechanism to score patient data based on current and past history.
